(19) United States
(12) Patent Application Publication
(10) Pub. No.: US 2010/0048555 A1
(43) Pub. Date: Feb. 25, 2010

(54) IMIDAZOTHIAZOLE DERIVATIVES AS MARK INHIBITORS

(76) Inventors: Ian Churcher, Royston (GB); Victoria Alexandra Doughty, Stansted Mountfitchet (GB); Peter Hunt, Horsham (GB); Matthew Stanton, Lansdale, PA (US)

Correspondence Address: MERCK AND CO., INC, P O BOX 2000, RAHWAY, NJ, 07065-0907, US

(21) Appl. No.: 12/374,535

(22) PCT Filed: Jul. 20, 2007
(86) PCT No.: PCT/GB2007/050426

§ 371 Date: Oct. 20, 2009

Related U.S. Application Data
(60) Provisional application No. 60/832,831, filed on Jul. 24, 2006.

(51) Int. Cl. A61K 31/5377; A61K 31/496; A61K 31/429; C07D 413/14; C07D 417/14; C07D 513/04; A61P 25/28

(52) U.S. Cl. 514/233.2; 514/254.02; 514/368; 544/133; 544/368; 548/154

(57) ABSTRACT

Compounds of formula (I): are inhibitors of MARK, and hence useful for treatment of disorders involving 10 hyperphosphorylation of tau.

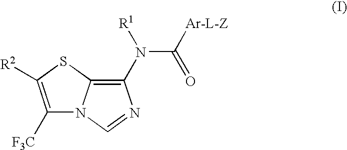

This invention relates to methods and materials for the treatment or prevention of neurodegenerative diseases such as Alzheimer's disease. In particular, there is disclosed a particular class of imidazothiazole derivatives which selectively inhibit microtubule affinity regulating kinase (MARK).

Alzheimer's disease (AD) is the most common cause of dementia in the elderly and is characterised by a decline in cognitive function, that progresses slowly and results in symptoms such as memory loss and disorientation. Death occurs, on average, 9 years after diagnosis. The incidence of AD increases with age, so that while about 5% of people over the age of 70 are sufferers, this figure increases to 20% of those over 80 years old.

Existing treatments exclusively target the primary symptoms of AD. Diseased neurons may release insufficient or excessive amounts of particular neurotransmitters, and so current drugs are aimed at increasing neurotransmitter levels or at reducing the stimulation of nerve cells by neurotransmitters. Although these drugs provide some improvement in the symptoms of AD, they fail to address the underlying cause of the disease.

The classic clinical and neuropathological features of AD consist of senile or neuritic plaques and tangled bundles of fibers (neurofibrillary tangles) [Verdile, G., et al, Pharm. Res. 50:397-409 (2004)]. In addition, there is a severe loss of neurons in the hippocampus and the cerebral cortex. Neuritic plaques are extracellular lesions, consisting mainly of deposits of β-amyloid peptide (Aβ), surrounded by dystrophic (swollen, damaged and degenerating) neurites and glial cells activated by inflammatory processes. In contrast, neurofibrillary tangles (NFTs) are intracellular clusters composed of a hyperphosphorylated form of the protein tau, which are found extensively in the brain (e.g. mainly in cortex and hippocampus in AD). Tau is a soluble cytoplasmic protein which has a role in microtubule stabilisation. Excessive phosphorylation of this protein renders it insoluble and leads to its aggregation into paired helical filaments, which in turn form NFTs.

The amyloid cascade hypothesis proposes that abnormal accumulation of Aβ peptides, particularly Aβ42, initiates a cascade of events leading to the classical symptoms of AD and ultimately, to the death of the patient. There is strong evidence [e.g. Rapoport, M., et al (2002) Proc. Natl. Acad. Sci. USA 99:6364-6369] that dysregulation of tau function is a key step in the cascade of Alzheimer's disease pathology leading ultimately to neuronal death. Furthermore, tau mutations and NFTs are found in other dementias in which Aβ pathology is absent, such as frontotemporal dementia, Pick's disease and parkinsonism linked to chromosome 17 (FTDP-17) [Mizutani, T. (1999) Rinsho Shikeigaku 39: 1262-1263]. Also, in AD the frequency of NFTs correlates to the degree of dementia better than that of senile plaques [Arriagada, P. V., et al (1992) Neurology 42:631-639], while significant numbers of amyloid plaques are often found in the brains of non-demented elderly people, suggesting that amyloid pathology on its own is not sufficient to cause dementia. For these reasons, normalisation of tau function (in particular prevention of hyperphosphorylation) is seen as a desirable therapeutic goal for the treatment of AD and other dementing conditions.

Tau is a 352-441 amino acid protein encoded by the Mapt (Microtubule-associated protein tau) gene which is widely expressed in the central nervous system (CNS) with localisation primarily in axons [Binder et al J. Cell Biol. 1985, 101(4), 1371-1378]. The major function of tau is regulation of the stability of microtubules (MTs), intracellular structural components comprised of tubulin dimers which are integral in regulating many essential cellular processes such as axonal transport and elongation as well as generation of cell polarity and shape. Tau binding to tubulin is a key factor in determining the rates of polymerisation/depolymerisation (termed dynamic instability) of MTs, and tau is therefore key to the regulation of many essential cellular processes [see, for example, Butner, K. A., Kirschner, M. W. (1991) J. Cell. Biol. 115: 717-730].

Tau is a basic protein with numerous serine and threonine residues, many of which are susceptible to phosphorylation. While normal tau has two to three phosphorylated amino acid residues, hyperphosphorylated tau found in AD and other tauopathies typically has eight or nine phosphorylated residues. A variety of kinases promote phosphorylation of these sites, including proline-directed kinases such as glycogen synthase kinase 313 (GSK313) and cyclin dependent kinase 5 (cdk5), and non-proline-directed kinases such as protein kinase A (PKA) and calmodulin (CaM) kinase II, which phosphorylate tau at Lys-(Ile/Cys)-Gly-Ser sequences, also known as KXGS motifs. One KXGS motif is found in each of the MT binding repeats. Phosphorylation at these sites is important for the regulation of tau-MT binding and while the degree of phosphorylation is normally low, it has been shown to be increased in brain tissue from AD patients. Phosphorylation of one particular residue within the KXGS motifs, Ser-262 has been shown to be elevated in tau protein extracted from the NFTs in AD [Hasegawa, M. et al (1992) J. Biol. Chem. 267:17047-17054] and phosphorylation at this site also appears to dramatically reduce MT binding [Biemat, J. et al. (1993) Neuron 11: 153-163].

Nishimura et al. [Cell 116: 671-682 (2004)] demonstrated that overexpression of the kinase PAR-1 in Drosophila led to enhanced tau-mediated toxicity and an increase in the phosphorylation of tau on Ser-262, Ser-356, and other amino acid residues, including sites phosphorylated by GSK313 and Cdk5. Their findings suggest that PAR-1 kinase acts as a master kinase during the process of tau hyperphosphorylation, with the phosphorylation of the Ser-262 and Ser-356 sites being a prerequisite for the subsequent phosphorylation at downstream sites by other kinases.

The mammalian ortho log of PAR-1 is microtubule affinity-regulating kinase (MARK). There are four MARK isoforms and these form part of the AMP-dependent protein kinase (AMPK) family. Like PAR-1, MARK is thought to phosphorylate tau, perhaps in response to an external insult, such as the disruption of Ca²⁺ homeostasis caused by Aβ, priming it for further phosphorylation events. It is not clear whether the phosphorylation of tau by MARK leads directly to its detachment from MTs or the subsequent phosphorylation events cause detachment. The resulting unbound, hyperphosphorylated tau is delocalised to the somatodendritic compartment and is then cleaved by caspases to form fragments prone to aggregation [Drewes, G. (2004). Trends Biochem. Sci 29:548-555; Gamblin, T. C., et al, (2003) Proc. Natl. Acad. Sci. U.S.A. 100:10032-10037]. These aggregates can grow into filaments, which are potentially toxic, eventually forming the NFTs found in AD.

For these reasons, it is proposed that MARK inhibitors will enable the prevention or amelioration of neurodegeneration in AD and other tauopathies.

The invention provides a compound of formula I:

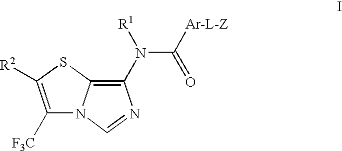

or a pharmaceutically acceptable salt or hydrate thereof, wherein:

R¹ represents H or C₁₋₄alkyl;

R² represents H, halogen C₁₋₄alkyl or CON(R⁴)₂;

each R⁴ independently represents H or C₁₋₄alkyl which is optionally substituted with up to 3 fluorine atoms;

Ar represents phenyl, naphthyl or heteroaryl of up to 10 ring atoms and is optionally substituted with halogen or C₁₋₄alkyl;

L represents a bond or a linking group represented by (X)ₘ—(CH₂)ₙ—(Y)ₚ;

m and p each independently is 0 or 1;

n is 0, 1, 2 or 3 but n is not 0 if m and p each represents 1;

X and Y independently represent O or NR³;

Z represents H, halogen, CF₃, CN, COR³, CO₂R³, CON(R³)₂, C₃₋₆cycloalkyl, phenyl, naphthyl or heterocyclyl of up to 10 ring atoms, said cycloalkyl, phenyl, naphthyl or heterocyclyl optionally bearing up to 2 substituents selected from halogen, C₁₋₄alkyl, CF₃, OH and C₁₋₄alkoxy;

with the proviso that when Z represents halogen or CN, L represents (X)ₘ—(CH₂)_{q} where q is 1, 2 or 3; and

R³ represents H or C₁₋₄alkyl; or two R³ groups attached to the same nitrogen atom may complete an N-heterocyclyl group of up to 10 ring atoms which optionally bears up to 2 substituents selected from halogen, C₁₋₄alkyl, CF₃ OH and C₁₋₄alkoxy.

In one embodiment, R² represents H, halogen or C₁₋₄alkyl, and the remaining variables are as defined above.

The invention further provides a pharmaceutical composition comprising a compound of formula I as defined above or a pharmaceutically acceptable salt or hydrate thereof and a pharmaceutically acceptable carrier.

The invention further provides a compound of formula I as defined above or a pharmaceutically acceptable salt or hydrate thereof for use in therapeutic treatment of humans or animals.

The invention further provides the use of a compound of formula I as defined above or a pharmaceutically acceptable salt or hydrate thereof for the manufacture of a medicament for treatment or prevention of a neurodegenerative disease associated with hyperphosphorylation of tau in a human patient.

There is also disclosed a method for treatment or prevention of a neurodegenerative disease associated with hyperphosphorylation of tau in a human patient, said method comprising administering to that patient an effective amount of a compound of formula I as defined above, or a pharmaceutically acceptable salt or hydrate thereof.

Neurodegenerative diseases associated with hyperphosphorylation of tau include AD, frontotemporal dementia, Pick's disease and parkinsonism linked to chromosome 17 (FTDP-17).

The invention further provides a compound of formula I as defined above, or a pharmaceutically acceptable salt or hydrate thereof, for use in reducing or preventing the hyperphosphorylation of tau in a human patient.

As used herein, the expression “C₁₋ₓalkyl” where x is an integer greater than 1 refers to straight-chained and branched alkyl groups wherein the number of constituent carbon atoms is in the range 1 to x. Particular alkyl groups are methyl, ethyl, n-propyl, isopropyl and t-butyl. Derived expressions such as “C₂₋₆alkenyl”, “hydroxyC₁₋₆alkyl”, “heteroarylC₁₋₆alkyl”, “C₂₋₆alkynyl” and “C₁₋₆alkoxy” are to be construed in an analogous manner. Most suitably, the number of carbon atoms in such groups is not more than 6.

The term “halogen” as used herein includes fluorine, chlorine, bromine and iodine of which fluorine and chlorine are preferred.

The expression “C₃₋₆cycloalkyl” as used herein refers to nonaromatic monocyclic hydrocarbon ring systems comprising from 3 to 6 ring atoms. Examples include cyclopropyl, cyclobutyl, cyclopentyl and cyclohexyl.

The term “heterocyclyl” as used herein refers to a ring system in which at least one of the ring atoms is N, O or S. Depending on the maximum number of ring atoms permitted, said ring system may be mono- or bicyclic. Any ring comprised by said system may saturated or unsaturated to any degree, including aromatic unless indicated otherwise. Attachment may be via any available ring atom unless indicated otherwise. “N-heterocyclyl” indicates attachment via a ring nitrogen and “C-heterocyclyl” indicates attachment via a ring carbon.

The term “heteroaryl” refers to heterocyclic groups in which at least one ring comprising a heteroatom is aromatic.

For use in medicine, the compounds of formula I may be in the form of pharmaceutically acceptable salts. Other salts may, however, be useful in the preparation of the compounds of formula I or of their pharmaceutically acceptable salts. Suitable pharmaceutically acceptable salts of the compounds of this invention include acid addition salts which may, for example, be formed by mixing a solution of the compound according to the invention with a solution of a pharmaceutically acceptable acid such as hydrochloric acid, sulphuric acid, methanesulphonic acid, benzenesulphonic acid, fumaric acid, maleic acid, succinic acid, acetic acid, benzoic acid, oxalic acid, citric acid, tartaric acid, carbonic acid or phosphoric acid. Alternatively, where the compound of the invention carries an acidic moiety, a pharmaceutically acceptable salt may be formed by neutralisation of said acidic moiety with a suitable base. Examples of pharmaceutically acceptable salts thus formed include alkali metal salts such as sodium or potassium salts; ammonium salts; alkaline earth metal salts such as calcium or magnesium salts; and salts formed with suitable organic bases, such as amine salts (including pyridinium salts) and quaternary ammonium salts.

When the compounds useful in the invention have one or more asymmetric centres, they may accordingly exist as enantiomers. Where the compounds according to the invention possess two or more asymmetric centres, they may additionally exist as diastereoisomers. It is to be understood that all such isomers and mixtures thereof in any proportion are encompassed within the scope of the present invention.

When a compound useful in the invention is capable of existing in tautomeric keto and enol forms, both of said forms are considered to be within the scope of the invention.

A nitrogen atom forming part of a heteroaryl ring may be in the form of the N-oxide. A sulphur atom forming part of a nonaromatic heterocycle may be in the form of the S-oxide or S,S-dioxide.

A heteroaryl group may be attached to the remainder of the molecule via a ring carbon or a ring nitrogen, provided that this is consistent with preservation of aromaticity.

In the compounds of formula I, R¹ is H or C₁₋₄alkyl, such as methyl, ethyl, n-propyl or isopropyl. In a particular embodiment R¹ is H.

R² represents H, halogen (preferably F or Cl), C₁₋₄alkyl (such as methyl, ethyl, n-propyl or isopropyl), or CON(R⁴)₂ where each R⁴ independently represents H or C₁₋₄ alkyl which is optionally substituted with up to 3 fluorine atoms. Particular identities for R² include H, CONH₂, CONHMe, CONHEt, CONHCH₂CF₃ and CONMe₂. In a particular embodiment R² is H.

Ar represents phenyl, naphthyl or heteroaryl of up to 10 ring atoms, any of which may bear a substituent selected from halogen and C₁₋₄alkyl (in addition to the -L-Z moiety). Examples of heteroaryl groups represented by Ar include 5-membered and 6-membered rings and benzo-fused analogues thereof, in particular 6-membered rings such as pyridine and pyrimidine. Examples of 5-membered heteroaryl rings represented by Ar include furan, thiophene and pyrazole. Ar may also represent a fused bicyclic heteroaromatic system in which both rings comprise one or more heteroatoms, such as 1-H-thieno[2,3-c]pyrazole. In a particular embodiment, Ar represents optionally substituted phenyl or pyridyl, and in a further embodiment Ar represents phenyl.

L represents a bond or a linking group represented by (X)ₘ—(CH₂)ₙ—(Y)ₚ where X, Y, m, n and p are as defined previously. Examples of linking groups represented by L include O, NH, N(CH₃), CH₂, OCH₂, CH₂O, NHCH₂, CH₂NH, CH₂CH₂, OCH₂CH₂, NHCH₂CH₂, CH₂CH₂CH₂, OCH₂CH₂O, and NHCH₂CH₂NH. In a particular embodiment, L is selected from a bond, O, NH, CH₂, OCH₂CH₂ and NHCH₂CH₂.

Z represents H, halogen, CF₃, CN, COR³, CO₂R³, CON(R³)₂, C₃₋₆cycloalkyl, phenyl, naphthyl or heterocyclyl of up to 10 ring atoms, said cycloalkyl, phenyl, naphthyl or heterocyclyl optionally bearing up to 2 substituents selected from halogen, C₁₋₄alkyl, CF₃, OH and C₁₋₄alkoxy; with the proviso that when Z represents halogen or CN, L represents (X)ₘ—(CH₂)_{q} where q is 1, 2 or 3; where R³ represents H or C₁₋₄alkyl; or two R³ groups attached to the same nitrogen atom may complete an N-heterocyclyl group as defined previously. Typically, R³ represents H, methyl or ethyl, or two R³ groups attached to the same nitrogen atom complete an N-heterocyclyl group such as pyrrolidin-1-yl, piperidin-1-yl, morpholin-4-yl, piperazin-1-yl or 4-methylpiperazin-1-yl.

In one embodiment Z represents H, halogen (e.g. F or Cl), CN, COR³, CO₂R³ or optionally substituted phenyl or heterocyclyl (in particular nonaromatic heterocyclyl such as azetidinyl, pyrrolidinyl, piperidinyl, morpholinyl or piperazinyl).

Specific embodiments of the moiety L-Z include: methoxy, Cl, F, CHO, CO₂Me, OH, phenoxy, piperazin-1-yl, 4-methylpiperazin-1-yl, 2-(piperidin-1-yl)ethoxy, morpholin-4-yl, hydroxymethyl, (4-methylpiperazin-1-yl)methyl, (piperidin-1-yl)methyl, (morpholin-4-yl)methyl and N-(1-methylpiperidin-4-yl)aminomethyl. Further embodiments of L-Z include H and CN.

Although the moiety L-Z may be attached at any available position on Ar, it is preferably not ortho to the carbonyl group to which Ar is attached.

A subset of the compounds of formula I is defined by formula II:

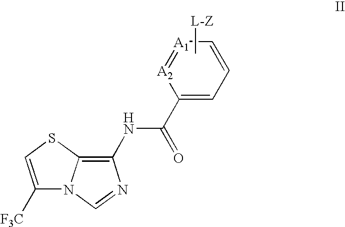

and pharmaceutically acceptable salts or hydrates thereof,

wherein A₁ and A₂ each represents CH or N but do not both represent N; and L and Z have the same meanings and specific identities as described previously.

In one embodiment, A₂ is CH. In a further embodiment, A₁ and A₂ are both CH.

Examples of compounds in accordance with formula II include those in which A₂ is CH and A₁, L and Z are as indicated in the following table, where “position” refers to the attachment point of L-Z relative to the carbonyl group:

| A₁ | L | Z | Position |
|---|---|---|---|
| CH | OCH₂ | H | para |
| CH | bond | Cl | para |
| CH | bond | F | para |
| CH | bond | 4-Me-piperazin-1-yl | para |
| CH | O | H | para |
| CH | OCH₂CH₂ | piperidin-1-yl | para |
| CH | bond | morpholin-1-yl | para |
| CH | bond | piperazin-1-yl | para |
| CH | CH₂O | H | para |
| CH | CH₂ | 4-Me-piperazin-1-yl | para |
| CH | CH₂ | piperidin-1-yl | para |
| CH | CH₂ | morpholin-4-yl | para |
| CH | OCH₂CH₂ | piperidin-1-yl | meta |
| CH | CH₂NH | 1-Me-piperidin-4-yl | para |
| CH | bond | Br | para |
| CH | O | Ph | para |
| CH | bond | CO₂Me | para |
| CH | O | H | meta |
| CH | bond | CHO | Para |
| N | bond | 4-Me-piperazin-1-yl | para |

Compounds of formula I may be obtained by coupling of an acid Z-L-Ar—CO₂H with an amine of formula (I):

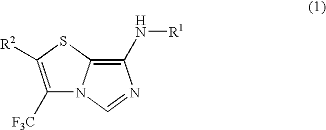

where L, Z, Ar, R¹ and R² have the same meanings as before. The reaction may be carried out using any of the standard techniques for amide bond formation, e.g. using carbonyl diimidazole as coupling agent in the presence of pyridine. Alternatively, the amines of formula (I) may be condensed with acid chlorides Z-L-Ar—COCl, typically in an aprotic solvent such as ethyl acetate, optionally with addition of base to scavenge the HCl released.

Amines (1) in which R¹ is H are obtainable by reduction of the nitro derivatives (2):

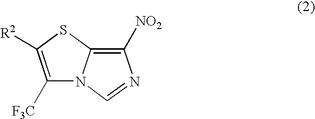

e.g. by hydrogenation over Pd/C. Amines (1) in which R¹ is C₁₋₄alkyl may be obtained by alkylation of the primary amines by standard methods, e.g. treatment with the appropriate aldehyde and sodium triacetoxyborohydride.

Nitro derivatives (2) are obtainable by reaction of the nitroimidazolethiol (3):

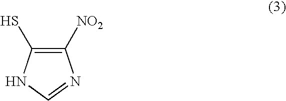

with CF₃COCH(R²)Br under the conditions described in Phosphorus, Sulfur and Silicon and the Related Elements (1989), 44, 203-7. Compound (3) may be obtained as described in Khimiya Geterotsiklicheskikh Soedinenii (1982), (6), 812-16.

Compounds of formula I in which R² represents CON(R⁴)₂ are most conveniently prepared by hydrolysis of the corresponding esters in which R² represents CO₂Me or CO₂Et, followed by coupling of the resulting carboxylic acid with (R⁴)₂NH using any of the known amide coupling techniques. The relevant esters are available via a modified form of the route described above involving reaction of the thiol (3) with CF₃COCH(Br)CO₂Et (or with the corresponding methyl ester).

Individual compounds in accordance with formula I may be converted to different compounds, also in accordance with formula I, using well known techniques of organic synthesis. For example, a compound of formula I in which L-Z represents F may be subjected to microwave heating (e.g. at 220° C. in N-methylpyrrolidone) with a suitable N-containing heterocycle to form the corresponding compound of formula I in which L-Z represents N-heterocyclyl. Alternatively, the fluorine atom may be similarly displaced by CN, and if desired the resulting nitrile converted to the corresponding amide. Likewise, a compound of formula I in which L-Z represents alkoxycarbonyl may be reduced (e.g. using DIBAL-H) to the corresponding compound where L-Z represents CH₂OH. This in turn may be oxidised (e.g. using Dess-Martin periodinane) to the corresponding aldehyde, which may be used in the reductive alkylation of an amine or suitable N-heterocycle. Also, a compound of formula I in which L-Z represents OH may be reacted with an alkanol of formula HO(CH₂)ₙ—Z (e.g. in THF solution in the presence of Ph₃P and a dialkylazodicarboxylate) to provide the corresponding compound containing a O(CH₂)ₙ—Z moiety, where Z has the same meaning as before.

Where they are not themselves commercially available, the starting materials and reagents described above may be obtained from commercially available precursors by means of well known synthetic procedures and/or the methods disclosed in the Examples section herein.

Where the above-described processes for the preparation of the compounds of use in the invention give rise to mixtures of stereoisomers, these isomers may be separated by conventional techniques such as preparative chromatography. The compounds may be prepared in racemic form, or individual enantiomers may be prepared either by enantiospecific synthesis or by resolution. The compounds may, for example, be resolved into their component enantiomers by standard techniques such as preparative HPLC, or the formation of diastereomeric pairs by salt formation with an optically active acid, such as di-p-toluoyl-D-tartaric acid and/or di-p-toluoyl-L-tartaric acid, followed by fractional crystallization and regeneration of the free base. The compounds may also be resolved by formation of diastereomeric esters or amides, followed by chromatographic separation and removal of the chiral auxiliary.

During any of the above synthetic sequences it may be necessary and/or desirable to protect sensitive or reactive groups on any of the molecules concerned. This may be achieved by means of conventional protecting groups, such as those described in Protective Groups in Organic Chemistry, ed. J. F. W. McOmie, Plenum Press, 1973; and T. W. Greene & P. G. M. Wuts, Protective Groups in Organic Synthesis, John Wiley & Sons, 1991. The protecting groups may be removed at a convenient subsequent stage using methods known from the art.

The compounds of formula I are suitably administered to patients in the form a pharmaceutical composition comprising the active ingredient (i.e. the compound of formula I or pharmaceutically acceptable salt or hydrate thereof) and a pharmaceutically acceptable carrier.

Preferably these compositions are in unit dosage forms such as tablets, pills, capsules, powders, granules, sterile parenteral solutions or suspensions, metered aerosol or liquid sprays, drops, ampoules, transdermal patches, auto-injector devices or suppositories; for oral, parenteral, intranasal, sublingual or rectal administration, or for administration by inhalation or insufflation. The principal active ingredient typically is mixed with a pharmaceutical carrier, e.g. conventional tableting ingredients such as corn starch, lactose, sucrose, sorbitol, talc, stearic acid, magnesium stearate and dicalcium phosphate, or gums, dispersing agents, suspending agents or surfactants such as sorbitan monooleate and polyethylene glycol, and other pharmaceutical diluents, e.g. water, to form a homogeneous preformulation composition containing a compound of the present invention, or a pharmaceutically acceptable salt thereof. When referring to these preformulation compositions as homogeneous, it is meant that the active ingredient is dispersed evenly throughout the composition so that the composition may be readily subdivided into equally effective unit dosage forms such as tablets, pills and capsules. This preformulation composition is then subdivided into unit dosage forms of the type described above containing from 0.1 to about 500 mg of the active ingredient of the present invention. Typical unit dosage forms contain from 1 to 100 mg, for example 1, 2, 5, 10, 25, 50 or 100 mg, of the active ingredient. Tablets or pills of the composition can be coated or otherwise compounded to provide a dosage form affording the advantage of prolonged action. For example, the tablet or pill can comprise an inner dosage and an outer dosage component, the latter being in the form of an envelope over the former. The two components can be separated by an enteric layer which serves to resist disintegration in the stomach and permits the inner component to pass intact into the duodenum or to be delayed in release. A variety of materials can be used for such enteric layers or coatings, such materials including a number of polymeric acids and mixtures of polymeric acids with such materials as shellac, cetyl alcohol and cellulose acetate.

The liquid forms in which the compositions useful in the present invention may be incorporated for administration orally or by injection include aqueous solutions, liquid- or gel-filled capsules, suitably flavoured syrups, aqueous or oil suspensions, and flavoured emulsions with edible oils such as cottonseed oil, sesame oil, coconut oil or peanut oil, as well as elixirs and similar pharmaceutical vehicles. Suitable dispersing or suspending agents for aqueous suspensions include synthetic and natural gums such as tragacanth, acacia, alginate, dextran, sodium carboxymethylcellulose, methylcellulose, poly(ethylene glycol), poly(vinylpyrrolidone) or gelatin.

In one embodiment of the invention, the compound of formula I is administered to a patient suffering from AD, FTDP-17, Pick's disease or frontotemporal dementia, preferably AD.

In an alternative embodiment of the invention, the compound of formula I is administered to a patient suffering from mild cognitive impairment or age-related cognitive decline. A favourable outcome of such treatment is prevention or delay of the onset of AD. Age-related cognitive decline and mild cognitive impairment (MCI) are conditions in which a memory deficit is present, but other diagnostic criteria for dementia are absent (Santacruz and Swagerty, American Family Physician, 63 (2001), 703-13). (See also “The ICD-10 Classification of Mental and Behavioural Disorders”, Geneva: World Health Organisation, 1992, 64-5). As used herein, “age-related cognitive decline” implies a decline of at least six months' duration in at least one of: memory and learning; attention and concentration; thinking; language; and visuospatial functioning and a score of more than one standard deviation below the norm on standardized neuropsychologic testing such as the MMSE. In particular, there may be a progressive decline in memory. In the more severe condition MCI, the degree of memory impairment is outside the range considered normal for the age of the patient but AD is not present. The differential diagnosis of MCI and mild AD is described by Petersen et al., Arch. Neurol., 56 (1999), 303-8. Further information on the differential diagnosis of MCI is provided by Knopman et al., Mayo Clinic Proceedings, 78 (2003), 1290-1308. In a study of elderly subjects, Tuokko et al (Arch, Neurol., 60 (2003) 577-82) found that those exhibiting MCI at the outset had a three-fold increased risk of developing dementia within 5 years.

Grundman et al (J. Mol. Neurosci., 19 (2002), 23-28) report that lower baseline hippocampal volume in MCI patients is a prognostic indicator for subsequent AD. Similarly, Andreasen et al (Acta Neurol. Scand, 107 (2003) 47-51) report that high CSF levels of total tau, high CSF levels of phospho-tau and lowered CSF levels of Aβ42 are all associated with increased risk of progression from MCI to AD.

Within this embodiment, the compound of formula I is advantageously administered to patients who suffer impaired memory function but do not exhibit symptoms of dementia. Such impairment of memory function typically is not attributable to systemic or cerebral disease, such as stroke or metabolic disorders caused by pituitary dysfunction. Such patients may be in particular people aged 55 or over, especially people aged 60 or over, and preferably people aged 65 or over. Such patients may have normal patterns and levels of growth hormone secretion for their age. However, such patients may possess one or more additional risk factors for developing Alzheimer's disease. Such factors include a family history of the disease; a genetic predisposition to the disease; elevated serum cholesterol; and adult-onset diabetes mellitus.

In a particular embodiment of the invention, the compound of formula I is administered to a patient suffering from age-related cognitive decline or MCI who additionally possesses one or more risk factors for developing AD selected from: a family history of the disease; a genetic predisposition to the disease; elevated serum cholesterol; adult-onset diabetes mellitus; elevated baseline hippocampal volume; elevated CSF levels of total tau; elevated CSF levels of phospho-tau; and lowered CSF levels of Aβ(1-42).

A genetic predisposition (especially towards early onset AD) can arise from point mutations in one or more of a number of genes, including the APP, presenilin-1 and presenilin-2 genes. Also, subjects who are homozygous for the ε4 isoform of the apolipoprotein E gene are at greater risk of developing AD.

The patient's degree of cognitive decline or impairment is advantageously assessed at regular intervals before, during and/or after a course of treatment in accordance with the invention, so that changes therein may be detected, e.g. the slowing or halting of cognitive decline. A variety of neuropsychological tests are known in the art for this purpose, such as the Mini-Mental State Examination (MMSE) with norms adjusted for age and education (Folstein et al., J. Psych. Res., 12 (1975), 196-198, Anthony et al., Psychological Med., 12 (1982), 397-408; Cockrell et al., Psychopharmacology, 24 (1988), 689-692; Crum et al., J. Am. Med. Assoc'n. 18 (1993), 2386-2391). The MMSE is a brief quantitative measure of cognitive status in adults. It can be used to screen for cognitive decline or impairment, to estimate the severity of cognitive decline or impairment at a given point in time, to follow the course of cognitive changes in an individual over time, and to document an individual's response to treatment. Another suitable test is the Alzheimer Disease Assessment Scale (ADAS), in particular the cognitive element thereof (ADAS-cog) (See Rosen et al., Am. J. Psychiatry, 141 (1984), 1356-64).

For treating or preventing Alzheimer's disease, a suitable dosage level is about 0.01 to 250 mg/kg per day, preferably about 0.01 to 100 mg/kg per day, and more preferably about 0.05 to 50 mg/kg of body weight per day, of the active compound. The compounds may be administered on a regimen of 1 to 4 times per day. In some cases, however, a dosage outside these limits may be used.

The compound of formula I optionally may be administered in combination with one or more additional compounds known to be useful in the treatment or prevention of AD or the symptoms thereof. Such additional compounds thus include cognition-enhancing drugs such as acetylcholinesterase inhibitors (e.g. donepezil and galanthamine), NMDA antagonists (e.g. memantine) or PDE4 inhibitors (e.g. Ariflo™ and the classes of compounds disclosed in WO 03/018579, WO 01/46151, WO 02/074726 and WO 02/098878). Such additional compounds also include cholesterol-lowering drugs such as the statins, e.g. simvastatin. Such additional compounds similarly include compounds known to modify the production or processing of Aβ in the brain (“amyloid modifiers”), such as compounds which modulate the secretion of Aβ (including γ-secretase inhibitors, γ-secretase modulators and β-secretase inhibitors), compounds which inhibit the aggregation of Aβ, and antibodies which selectively bind to Aβ. Such additional compounds further include growth hormone secretagogues, e.g. as described in WO 2004/080459.

In this embodiment of the invention, the amyloid modifier may be a compound which inhibits the secretion of Aβ, for example an inhibitor of γ-secretase (such as those disclosed in WO 01/53255, WO 01/66564, WO 01/70677, WO 01/90084, WO 01/77144, WO 02/30912, WO 02/36555, WO 02/081435, WO 02/081433, WO 03/018543, WO 03/013506, WO 03/013527, WO 03/014075, WO 03/093251, WO 03/093252, WO 03/093253, WO 03/093264, WO 2004/031137, WO 2004/031138, WO 2004/031139, WO 2004/039370, WO 2004/039800, WO 2004/101538, WO 2004/101539 and WO 2005/030731), or a β-secretase inhibitor (such as those disclosed in WO 03/037325, WO 03/030886, WO 03/006013, WO 03/006021, WO 03/006423, WO 03/006453, WO 02/002122, WO 01/70672, WO 02/02505, WO 02/02506, WO 02/02512, WO 02/02520, WO 02/098849 and WO 02/100820), or any other compound which inhibits the formation or release of Aβ including those disclosed in WO 98/28268, WO 02/47671, WO 99/67221, WO 01/34639, WO 01/34571, WO 00/07995, WO 00/38618, WO 01/92235, WO 01/77086, WO 01/74784, WO 01/74796, WO 01/74783, WO 01/60826, WO 01/19797, WO 01/27108, WO 01/27091, WO 00/50391, WO 02/057252, US 2002/0025955 and US2002/0022621.

Within this embodiment, the amyloid modifier is advantageously a γ-secretase inhibitor, preferred examples of which include a compound of formula XI:

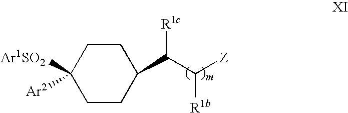

wherein the variables are as defined in WO 03/018543. Preferred examples include those defined by formula XIa:

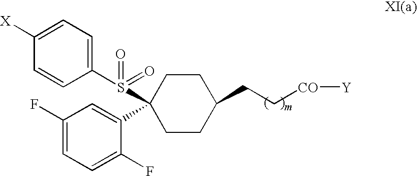

and the pharmaceutically acceptable salts thereof, wherein m is 0 or 1, X is C₁ or CF₃, and Y is OH, OC₁₋₆alkyl, NH₂ or NHC₁₋₆alkyl. Particular examples include those in which m is 1 and Y is OH (or the sodium salts thereof), and those in which m is 0 and Y is NH₂ or NHC₁₋₆alkyl.

Another preferred class of γ-secretase inhibitors for use in this embodiment of the invention is that defined by formula XII:

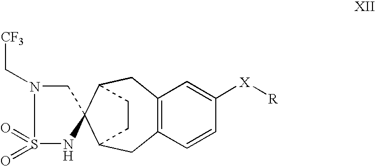

and the pharmaceutically acceptable salt thereof,
wherein the variables are as defined in WO 03/093252.

X is very aptly 5-substituted-thiazol-2-yl, 5-substituted-4-methylthiazol-2-yl, 5-substituted-1-methylpyrazol-3-yl, 1-substituted-imidazol-4-yl or 1-substituted-1,2,4-triazol-3-yl. Preferably, R represents optionally-substituted phenyl or heteroaryl such as phenyl, monohalophenyl, dihalophenyl, trihalophenyl, cyanophenyl, methylphenyl, methoxyphenyl, trifluoromethylphenyl, trifluoromethoxyphenyl, pyridyl, monohalopyridyl and trifluoromethylpyridyl, wherein “halo” refers to fluoro or chloro. Particularly preferred identities of R—X— include 5-(4-fluorophenyl)-1-methylpyrazol-3-yl, 5-(4-chlorophenyl)-1-methylpyrazol-3-yl and 1-(4-fluorophenyl)imidazol-4-yl. Such compounds may be prepared by methods disclosed in WO 03/093252.

Alternatively, the amyloid modifier may be a compound which modulates the action of 7-secretase so as to selectively attenuate the production of Aβ(1-42). Compounds reported to show this effect include certain non-steroidal antiinflammatory drugs (NSAIDs) and their analogues (see WO 01/78721 and US 2002/0128319 and Weggen et al Nature, 414 (2001) 212-16; Morihara et al, J. Neurochem., 83 (2002), 1009-12; and Takahashi et al, J. Biol. Chem., 278 (2003), 18644-70), and compounds which modulate the activity of PPARα and/or PPARδ (WO 02/100836). Further examples of γ-secretase modulators are disclosed in WO 2006/008558, WO 2006/043064, WO 2005/054193, WO 2005/013985 and WO 2005/108362.

Alternatively, the amyloid modifier may be a compound which inhibits the aggregation of Aβ. Suitable examples include chelating agents such as clioquinol (Gouras and Beal, Neuron, 30 (2001), 641-2) and the compounds disclosed in WO 99/16741, in particular that known as DP-109 (Kalendarev et al, J. Pharm. Biomed. Anal., 24 (2001), 967-75). Other inhibitors of Aβ aggregation suitable for use in the invention include the compounds disclosed in WO 96/28471, WO 98/08868 and WO 00/052048, including the compound known as Apan™ (Praecis); WO 00/064420, WO 03/017994, WO 99/59571 and the compound known as Alzhemed™ (Neurochem); WO 00/149281 and the compositions known as PTI-777 and PTI-00703 (ProteoTech); WO 96/39834, WO 01/83425, WO 01/55093, WO 00/76988, WO 00/76987, WO 00/76969, WO 00/76489, WO 97/26919, WO 97/16194, and WO 97/16191. Further examples include phytic acid derivatives as disclosed in U.S. Pat. No. 4,847,082 and inositol derivatives as taught in US 2004/0204387.

Alternatively, the amyloid modifier may be an antibody which binds selectively to Aβ. Said antibody may be polyclonal or monoclonal, but is preferably monoclonal, and is preferably human or humanized. Preferably, the antibody is capable of sequestering soluble Aβ from biological fluids, as described in WO 03/016466, WO 03/016467, WO 03/015691 and WO 01/62801. Suitable antibodies include humanized antibody 266 (described in WO 01/62801) and the modified version thereof described in WO 03/016466. Suitable antibodies also include those specific to Aβ-derived diffusible ligands (ADDLS), as disclosed in WO 2004/031400.

As used herein, the expression “in combination with” requires that therapeutically effective amounts of both the compound of formula I and the additional compound are administered to the subject, but places no restriction on the manner in which this is achieved. Thus, the two species may be combined in a single dosage form for simultaneous administration to the subject, or may be provided in separate dosage forms for simultaneous or sequential administration to the subject. Sequential administration may be close in time or remote in time, e.g. one species administered in the morning and the other in the evening. The separate species may be administered at the same frequency or at different frequencies, e.g. one species once a day and the other two or more times a day. The separate species may be administered by the same route or by different routes, e.g. one species orally and the other parenterally, although oral administration of both species is preferred, where possible. When the additional compound is an antibody, it will typically be administered parenterally and separately from the compound of formula I.

In a further aspect, the invention provides the combination of a compound of formula I and a compound of formula XI(a) or a pharmaceutically acceptable salt thereof for use in treatment or prevention of Alzheimer's disease. Said use may involve the simultaneous or separate administration of the respective compounds to a patient in need of such treatment or prevention.

In a further aspect, the invention provides a pharmaceutical composition comprising, in a pharmaceutically acceptable carrier, a compound of formula I and a compound of formula XI(a) or a pharmaceutically acceptable salt thereof. Preferably, the pharmaceutical composition is in a unit dose form suitable for oral administration, such as a tablet or a capsule.

### EXAMPLES

### MARK 3 Assay

MARK3 activity was assayed in vitro using a Cdc25C biotinylated peptide substrate (Cell Signalling Technologies). The phosphopeptide product was quantitated using a Homogenous Time-Resolved Fluorescence (HTRF) assay system (Park et al., 1999, Anal. Biochem. 269:94-104). The reaction mixture contained 50 mM HEPES/Tris-HCl, pH 7.4; 10 mM NaCl, 5 mM MgCl₂, 0.2 mM NaVO₄, 5 mM β-glycerol phosphate, 0.1% Tween-20, 2 mM dithiothreitol, 0.1% BSA, 10 μM ATP, 1 μM peptide substrate, and 10 nM recombinant MARK3 enzyme (University of Dundee) in a final volume of 12 μl. The buffer additionally contained protease inhibitor cocktail (Roche EDTA-free, 1 tab per 50 ml). The kinase reaction was incubated for 2 hours at 25° C., and then terminated with 3 μl Stop/Detection Buffer (50 mM HEPES, pH 7.0, 16.6 mM EDTA, 0.5M KF, 0.1% Tween-20, 0.1% BSA, 2 μg/ml SLX^{ent} 665 (CISBIO), and 2 μg/ml Eu³⁺ cryptate label antibody (CISBIO)). The reaction was allowed to equilibrate overnight at 0° C., and relative fluorescent units were read on an HTRF enabled plate reader (e.g. TECAN GENios Pro). Inhibitor compounds were assayed in the reaction described above to determine compound IC50s. Aliquots of compound dissolved in DMSO were added to the reaction wells in a third-log dilution series covering a range of 1 nM to 10 μM. Relative phospho substrate formation, read as HTRF fluorescence units, was measured over the range of compound concentrations and a titration curve generated.

The compounds listed below gave IC50 values of 1 μM or less, typically 500 nM or less, in the above assay.

More specifically, Examples 2, 13, 14 and 19 gave values in the range 500 nM-1 μM; Examples 1, 7, 9, 14, 16, 17 and 18 gave values in the range 100-500 nM; and Examples 8 and 12 gave values of less than 100 nM.

### Intermediate 1

### 3-(Trifluoromethyl)imidazo[5,1-b][1,3]thiazol-7-amine

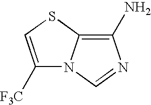

7-nitro-3-(trifluoromethyl)imidazo[5,1-b][1,3]thiazole (2.77 g, 11.66 mmol) was dissolved in ethyl acetate (50 ml) in a Parr vessel and the flask flushed with N₂. Pd—C (1.241 g, 1.166 mmol) was then added and the flask evacuated and flushed with N₂ three times. The flask was the evacuated and flushed with H₂ three times and then shaken at 3.5 bar for 2 h. The reaction mixture was then filtered through catalyst filter paper, washing with ethyl acetate (10 ml) and the mixture taken forward directly into the next step without concentration.

### Example 1

### 4-Fluoro-N-[3-(trifluoromethyl)imidazo[5,1-b][1,3]thiazol-7-yl]benzamide

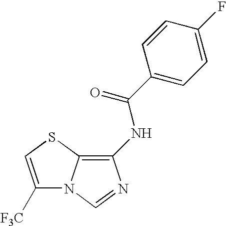

Triethylamine (0.882 ml, 6.33 mmol) was added to a solution of 3-(trifluoromethyl)imidazo[5,1-b][1,3]thiazol-7-amine (2.11 mmol) in ethyl acetate (10 ml). Then 4-fluorobenzoyl chloride (0.324 ml, 2.74 mmol) was added and the reaction allowed to stir for 16 h. Water (10 ml) was added and the mixture was extracted with ethyl acetate. The combined organic fractions were washed with water (10 ml), dried (Na₂SO₄), filtered and the solvent was evaporated under reduced pressure. The mixture was then triturated with MeOH to give a pale pink solid (386 mg). The water phase still contained some pink solid, so this was filtered. This solid was then triturated with methanol and ether to give a cream solid (95 mg). ¹H NMR δ (ppm) (DMSO): 11.28 (1H, s), 8.21 (1H, s), 8.13 (3H, dd, J=4.9, 8.2 Hz), 7.34 (2H, t, J=8.8 Hz); m/z (ES⁺) 330 (M+H⁺).

### Example 2

### 4-Hydroxy-N-[3-(trifluoromethyl)imidazo[5,1-b][1,3]thiazol-7-yl]benzamide

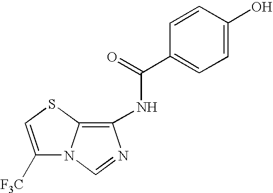

4-Hydroxybenzoic acid (291 mg, 2.11 mmol) was dissolved in pyridine (4 ml) and carbonyl diimidazole (342 mg, 2.11 mmol) added. After 2 hours, Intermediate 1 (2.11 mmol) as a solution in ethyl acetate (5 ml) was added, and the reaction mixture stirred for 16 h. The mixture was concentrated in vacuo then preadsorbed onto silica gel. The residue was purified by column chromatography on silica gel, eluting with 20-50% EtOAc/isohexane to give the title compound as a yellow solid. Trituration with Et₂O, MeOH, hexane mixtures gave the title compound as a cream solid (275 mg, 39%); ¹H NMR δ(ppm) (DMSO): 10.92 (1H, s), 10.16 (1H, s), 8.17 (1H, s), 8.12 (1H, s), 7.96 (2H, t, J=12.3 Hz), 6.83 (2H, d, J=8.7 Hz); m/z (ES⁺) 328 (M+H⁺).

### Examples 3-6

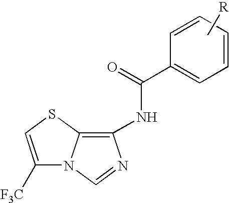

Following the procedures of Example 1 or Example 2, using the appropriate benzoyl chloride or benzoic acid, the following were prepared:

| Example | R | m/z (ES⁺) (M + H⁺) | Procedure |
|---|---|---|---|
| 3 | 4-bromo | 390, 392 | 1 |
| 4 | 4-phenoxy | 418 | 2 |
| 5 | 4-carboxylic acid methyl | 370 | 1 |
| | ester | | |
| 6 | 3-hydroxy | 328 | 2 |

### Example 7

### 4-Chloro-N-[3-(trifluoromethyl)imidazo[5,1-b][1,3]thiazol-7-yl]benzamide

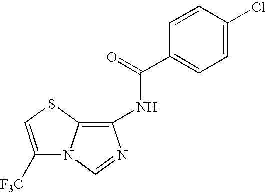

The procedures from Intermediate 1 and Example 1 were followed, using DMF instead of ethyl acetate as solvent, and 4-chlorobenzoyl chloride instead of 4-fluorobenzoyl chloride; ¹H NMR δ (ppm)(CDCl₃): 9.29 (1H, s), 7.87 (2H, d, J=8.5 Hz), 7.70 (1H, s), 7.47 (2H, d, J=8.5 Hz); m/z (ES⁺) 346 (M+H⁺).

### Example 8

### 4-(4-Methylpiperazin-1-yl)-N-[3-(trifluoromethyl)imidazo[5,1-b][1,3]thiazol-7-yl]benzamide

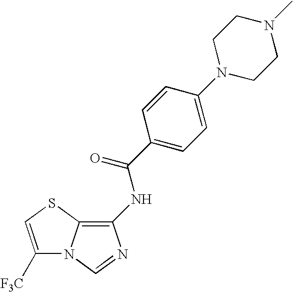

N-methyl piperazine (0.202 ml, 1.822 mmol) was added to a solution of 4-fluoro-N-[3-(trifluoromethyl)imidazo[5,1-b][1,3]thiazol-7-yl]benzamide (Example 1) (100 mg, 0.304 mmol) in NMP (2 ml). The reaction was heated by microwave for 1 h at 225° C. The NMP (2 ml) was removed in vacuo, then the brown oil taken up in MeOH, and absorbed onto an SCX cartridge, washing with MeOH, and eluting the product with 2M NH₃ in MeOH. The brown oil was then subjected to Agilent Purification, High pH, Sunfire column, 30-55% gradient. The HPLC fractions were concentrated in vacuo, then the residue extracted with ethyl acetate, dried over Na₂SO₄ then concentrated in vacuo. Trituration with ether gave the title compound as a pale brown solid (7.5 mg, 6%); ¹H NMR δ (ppm) (DMSO): 10.86 (1H, s), 8.16 (1H, s), 8.11 (1H, s), 7.94 (2H, d, J=8.5 Hz), 6.97 (2H, d, J=8.5 Hz), 2.50 (4H, obs), 2.45-2.41 (4H, m), 2.22 (3H, s); m/z (ES) 410 (M+H⁺).

### Examples 9-11

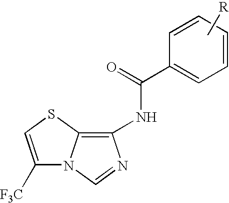

Following the procedures of Example 8, using the appropriate amine, the following were prepared:

| Example | R | m/z (ES⁺) (M + H⁺) |
|---|---|---|
| 9 | 4-N-morpholine | 397 |
| 10 | 4-N-piperidine | 395 |
| 11 | 4-N-piperazine | 396 |

### Example 12

### 4-(2-Piperidin-1-ylethoxy)-N-[3-(trifluoromethyl) imidazo[5,1-b][1,3]thiazol-7-yl]benzamide

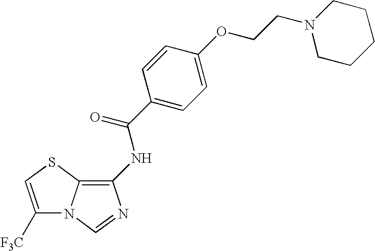

4-Hydroxy-N-[3-(trifluoromethyl)imidazo[5,1-b][1,3]thiazol-7-yl]benzamide (Example 2) (50 mg, 0.153 mmol) was dissolved in THF (5 ml) and triphenylphosphine (60.1 mg, 0.229 mmol) and 1-piperidine ethanol (0.030 ml, 0.229 mmol) added. Diisopropyl azodicarboxylate (0.045 ml, 0.229 mmol) was added, the mixture stirred for 48 hours, concentrated in vacuo, the residue dissolved in MeOH and absorbed onto an SCX cartridge, washing with MeOH, then eluting with 2M NH₃ in MeOH. After concentration in vacuo, the yellow solid was triturated with Et₂O, to give the title compound (20 mg, 30%) as a colourless solid; ¹H NMR δ (ppm) (DMSO): 11.03 (1H, s), 8.18 (1H, s), 8.13 (1H, s), 8.03 (2H, d, J=8.8 Hz), 7.03 (2H, d, J=8.8 Hz), 4.15 (2H, t, J=5.9 Hz), 2.67 (2H, t, J=5.8 Hz), 2.43 (4H, s), 1.50 (4H, s), 1.38 (2H, s); m/z (ES⁺) 439 (M+H⁺).

### Example 13

### 3-(2-Piperidin-1-ylethoxy)-N-[3-(trifluoromethyl) imidazo[5,1-b][1,3]thiazol-7-yl]benzamide

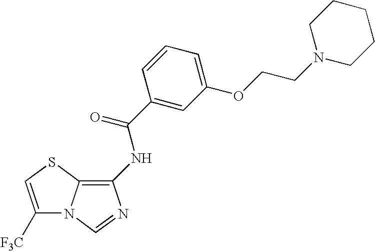

The procedure for Example 12 was used, starting with Example 6 instead of Example 2.

¹H NMR δ (ppm) (DMSO): 11.21 (1H, s), 8.20 (1H, s), 8.14 (1H, s), 7.64 (1H, s), 7.61 (1H, d, J=7.4 Hz), 7.39 (1H, t, J=8.0 Hz), 7.13 (1H, d, J=7.6 Hz), 4.15 (2H, t, J=5.9 Hz), 2.68 (2H, t, J=5.9 Hz), 2.47-2.43 (4H, m), 1.54-1.48 (4H, m), 1.41-1.35 (2H, m); m/z (ES⁺) 439 (M+H⁺).

### Example 14

### 4-(Hydroxymethyl)-N-[3-(trifluoromethyl)imidazo[5,1-b][1,3]thiazol-7-yl]benzamide

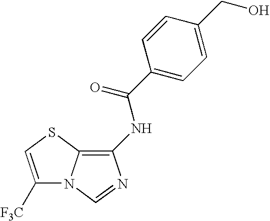

DIBAL-H in DCM (1M, 3.25 ml, 3.25 mmol) was added to a stirred mixture of methyl 4-({[3-(trifluoromethyl)imidazo[5,1-b][1,3]thiazol-7-yl]amino}carbonyl)benzoate (Example 5) (300 mg, 0.812 mmol) in DCM (40 ml) at −78° C. and the mixture was warmed to 0° C. and stirred for 1 h. After recooling to −78° C., MeOH (4 ml) and a 1.0M solution of Rochelle's salt (20 ml) were added, and the reaction allowed to warm to RT and stirred for 1 h 30 min. The resulting precipitate was isolated by filtration to give the title compound (235 mg, 85%) as a cream solid; ¹H NMR δ (ppm) (DMSO): 11.18 (1H, s), 8.20 (1H, s), 8.14 (1H, s), 8.01 (2H, d, J=8.3 Hz), 7.43 (2H, d, J=8.2 Hz), 5.32 (1H, t, J=5.6 Hz), 4.58 (2H, d, J=5.6 Hz); m/z (ES⁺) 342 (M+H⁺).

### Example 15

### 4-Formyl-N-[3-(trifluoromethyl)imidazo[5,1-b][1,3]thiazol-7-yl]benzamide

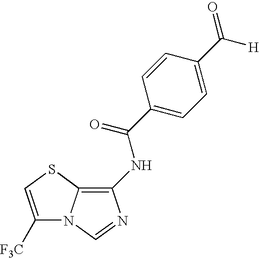

Dess-Martin Periodinane (298 mg, 0.703 mmol) was added to a stirred mixture of 4-(hydroxymethyl)-N-[3-(trifluoromethyl)imidazo[5,1-b][1,3]thiazol-7-yl]benzamide (Example 14) (160 mg, 0.469 mmol) in tetrahydrofuran and the mixture was stirred at room temperature for 3 h. A mixture of 1 N Na₂S₂O₃ and sat NaHCO₃ (1:7) was added and the reaction stirred for a further 1 h. The reaction was then filtered to give the title compound (122 mg, 77%) as a yellow solid; ¹H NMR δ (ppm) (DMSO): 11.50 (1H, s), 10.11 (1H, s), 8.24-8.20 (3H, m), 8.16 (1H, s), 8.02 (2H, d, J=8.0 Hz); m/z (ES) 340 (M+H⁺).

### Example 16

### 4-(Morpholin-4-ylmethyl)-N-[3-(trifluoromethyl)imidazo[5,1-b][1,3]thiazol-7-yl]benzamide

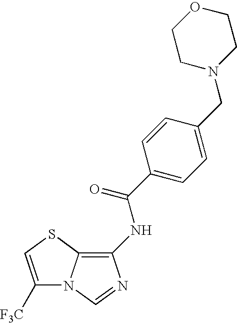

Sodium triacetoxyborohydride (94 mg, 0.442 mmol) was added to a stirred mixture of 4-formyl-N-[3-(trifluoromethyl)imidazo[5,1-b][1,3]thiazol-7-yl]benzamide (Example 15) (30 mg, 0.088 mmol) and morpholine (0.039 ml, 0.442 mmol) in 1,2-dichloroethane and the mixture was stirred at room temperature for 18 h. After 18 h the yellow colour of the starting material dissipated and a colourless suspension appeared. NaHCO₃ and DCM were added and the reaction stirred for 30 mins. The layers were separated and the aqueous phase extracted with DCM. The combined organic fractions were dried over Na₂SO₄ and concentrated in vacuo. The residue was purified by column chromatography on silica gel, eluting with EtOAc/isohexane to give the title compound (17 mg, 47%) as a colorless solid.

### Example 17

### 4-(Piperidin-1-ylmethyl)-N-[3-(trifluoromethyl)imidazo[5,1-b][1,3]thiazol-7-yl]benzamide

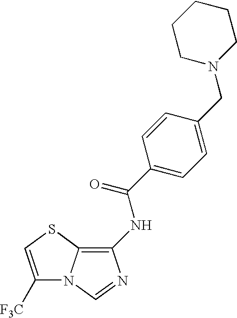

The procedure for Example 16 was used, employing piperidine instead of morpholine; ¹H NMR δ (ppm) (DMSO): 11.16 (1H, s), 8.19 (1H, s), 8.14 (1H, s), 8.00 (2H, d, J=8.1 Hz), 7.41 (2H, d, J=8.1 Hz), 3.49 (2H, s), 2.33 (4H, s), 1.51-1.49 (4H, m), 1.39 (2H, s).

### Example 18

### 4-[(4-methylpiperazin-1-yl)methyl]-N-[3-(trifluoromethyl) imidazo[5,1-b][1,3]thiazol-7-yl]benzamide

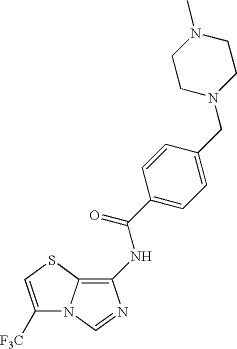

The procedure for Example 16 was used, employing N-methylpiperazine instead of morpholine; ¹H NMR δ (ppm) (DMSO): 11.18 (1H, s), 8.20 (1H, s), 8.14 (1H, s), 8.01 (2H, d, J=8.2 Hz), 7.41 (2H, d, J=8.2 Hz), 3.52 (2H, s), 2.38 (8H, s), 2.15 (3H, s).

### Example 19

### 6-(4-methylpiperazin-1-yl)-N-[3-(trifluoromethyl)imidazo[5,1-b][1,3]thiazol-7-yl]nicotinamide

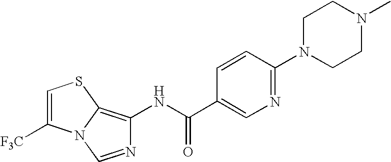

### Step 1: 6-chloro-N-[3-(trifluoromethyl)imidazo[5,1-b][1,3]thiazol-7-yl]nicotinamide

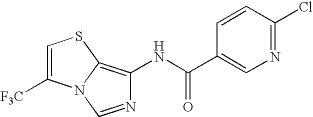

Triethylamine (0.9 mL, 6.33 mmol) was added to a solution of 3-(trifluoromethyl)-imidazo[5,1-b][1,3]thiazol-7-amine (intermediate 1) (2.11 mmol) in ethyl acetate (12.5 mL). 6-Chloro-nicotinoyl chloride (0.483 g, 2.74 mmol) was then added and the reaction mixture allowed to stir for 12 h at RT. Then the precipitate was filtered off and dried to give 6-chloro-N-[3-(trifluoromethyl)imidazo[5,1-b][1,3]thiazol-7-yl]nicotinamide.

### Step 2: 6-(4-methylpiperazin-1-yl)-N-[3-(trifluoromethyl)imidazo[5,1-b][1,3]thiazol-7-yl]nicotinamide

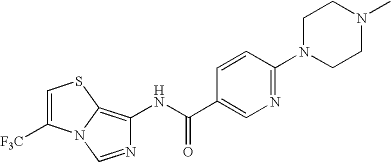

1-Methylpiperazine (0.032 mL, 0.29 mmol) was added to a solution of 6-chloro-N-[3-(trifluoromethyl)imidazo[5,1-b][1,3]thiazol-7-yl]nicotinamide (0.1 g, 0.29 mmol) in DMSO (1 mL) at RT in a stream of argon and the reaction mixture was stirred for 48 h. Then it stirred at 60° C. for 24 h. Then H₂O, aqueous saturated NaHCO₃ were added, the precipitate was filtered off, washed with Et₂O and dried. The precipitate was purified by chromatography (silica gel 63-100 μm, 4 mL, CHCl₃→CHCl₃:MeOH (85:15)) to furnish 6-(4-methylpiperazin-1-yl)-N-[3-(trifluoromethyl)imidazo[5,1-b][1,3]thiazol-7-yl]nicotinamide. ¹H NMR (400 MHz, DMSO-d₆): 10.96 (1H, s); 8.78 (1H, d, J₁=2.4 Hz); 8.10-8.18 (3H, m); 6.88 (1H, d, J₁=9.0 Hz); 3.60-3.68 (4H, m); 2.39-2.45 (4H, m); 2.24 (3H, s). LC-MS APCI: m/z 411.0 [M+H]⁺.

### Examples 20-26

Using analogous procedures, the following were also prepared:

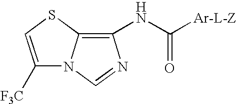

| Example | Ar—L—Z |
|---|---|
| 20 | 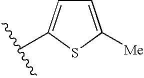 |
| 21 | 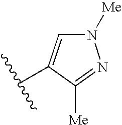 |
| 22 | 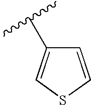 |
| 23 | 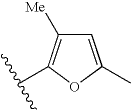 |
| 24 | 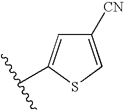 |
| 25 | 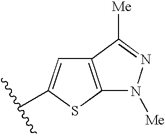 |
| 26 | 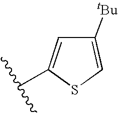 |

### Example 27

### 7-{[4-(4-methylpiperazin-1-yl)benzoyl]amino}-3-(trifluoromethyl)imidazo[5,1-b][1,3]thiazole-2-carboxamide

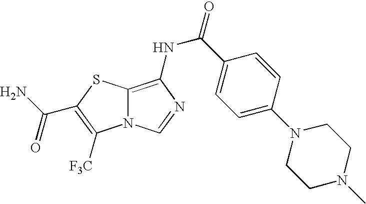

### Step 1: ethyl 2-bromo-4,4,4-trifluoro-3-oxobutanoate

To a solution of 10 g (8 mL, 54.35 mmol) of ethyl 4,4,4-trifluoro-3-oxobutanoate in 20 mL of CCL over a period of 1 h was added a solution of 8.69 g (2.8 mL, 54.35 mmol) of Br₂ in 30 mL in CCL. The reaction mixture was stirred at room temperature over a period of 16 h and evaporated in vacuum to give a crude product (˜8% of impurities). Yield: 73%.

### Step 2: ethyl 7-nitro-3-(trifluoromethyl)imidazo[5,1-b][1,3]thiazole-2-carboxylate

2.00 g (13.80 mmol) of 4-nitro-1H-imidazole-5-thiol was dissolved in 50 mL of DMF. Tri-n-butylphosphine (7.60 g, 0.55 mmol) and 3.63 mg (13.80 mmol) of ethyl 2-bromo-4,4,4-trifluoro-3-oxobutanoate were added. The reaction mixture was stirred over a period of 26 h and evaporated. The residue was subjected to coevaporation with xylene. POCl₃ (100 mL) was added, and the mixture was stirred overnight at 100-110° C. The product was purified by chromatography (chloroform-ethanol, 60:1). Yield: 422 mg (10%).

### Step 3: ethyl 7-amino-3-(trifluoromethyl)imidazo[5,1-b][1,3]thiazole-2-carboxylate

422 mg (1.36 mmol) of ethyl 7-nitro-3-(trifluoromethyl)imidazo[5,1-b][1,3]thiazole-2-carboxylate was dissolved in 10 mL of ethyl acetate. Then 500 mg of 10% Pd/C moistened with 0.5 g of H₂O was added. The reaction mixture was stirred in an atmosphere of hydrogen at 50 psi over a period of 5 h and filtered through Celite. The conversion degree was considered to be 90%. The crude product was carried into the next reaction as a solution due to its instability.

### Step 4: ethyl 7-{[4-(4-methylpiperazin-1-yl)benzoyl]amino}-3-(trifluoromethyl)imidazo[5,1-b][1,3]thiazole-2-carboxylate

To a solution of 1.36 mmol of ethyl 7-amino-3-(trifluoromethyl)imidazo[5,1-b][1,3]thiazole-2-carboxylate in 50 mL of ethyl acetate were added 0.76 mL (5.44 mmol) of triethylamine and 1.768 mmol of 4-(4-methylpiperazin-1-yl)benzoyl chloride. The reaction mixture was stirred over a period of 90 h and concentrated. The residue was suspended in water and filtered off. The residue on the filter was washed with water, then with ether. Yield: 420 mg (64%).

### Step 5: 7-{[4-(4-methylpiperazin-1-yl)benzoyl]amino}-3-(trifluoromethyl)imidazo[5,1-b][1,3]thiazole-2-carboxylic acid

To a solution of 419 mg (0.871 mmol) of ethyl 7-{[4-(4-methylpiperazin-1-yl)benzoyl]amino}-3-(trifluoromethyl)imidazo[5,1-b][1,3]thiazole-2-carboxylate in 5 mL of THF was added 13.0 mL (1.30 mmol) of 0.1M aqueous LiOH. The reaction mixture was stirred over a period of 12 h and neutralized to pH 7 with acetic acid. The formed precipitate was filtered off, washed with water, then with ether, and vacuum-dried. Yield: 281 mg (71%).

### Step 6: Amide Coupling

34 mg (75 umol) of 7-{[4-(4-methylpiperazin-1-yl)benzoyl]amino}-3-(trifluoromethyl)imidazo[5,1-b][1,3]thiazole-2-carboxylic acid was dissolved in 2 mL in DMF. DIPEA (1.87 g, 65 μL, 375 μmol) was added, and the mixture was stirred over a period of 5 min. Then BOP (43 mg, 97.5 μmol) was added. After 5 min 0.5M ammonia in dioxane (1.50 mL, 750 μmol) was added. After 10 h the mixture was evaporated. The residue was suspended in 20% potash, then in water. The formed precipitate was filtered off, washed on the filter with water, ether, and dichloromethane. Yield: 13.8 mg. The product was purified by preparative HPLC. ¹H NMR (400 MHz, DMSO-d₆): 2.25 (3H, s), 2.40-2.50 (4H, m), 3.27-3.37 (4H, m), 6.96 (2H, d, J=8.55 Hz), 7.95 (2H, d, J=7.35 Hz), 8.01-8.09 (1H, m), 8.08-8.13 (1H, m), 8.22-8.26 (1H, m), 10.68 (1H, br s). LC-MS APCI: m/z 453.5 [M+H]⁺.

The following examples were prepared in an analogous manner to that described in Example 27, using the appropriate amine in the final step.

| Name | Example | Structure | LCMS M + H = |
|---|---|---|---|
| N-methyl-7-{[4-(4- methylpiperazin-1- yl)benzoyl]amino}-3- (trifluoromethyl)imidazo[5,1- b][1,3]thiazole-2- carboxamide | 28 | 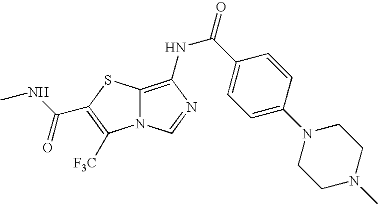 | 467.5 |
| N-ethyl-7-{[4-(4- methylpiperazin-1- yl)benzoyl]amino}-3- (trifluoromethyl)imidazo[5,1- b][1,3]thiazole-2- carboxamide | 29 | 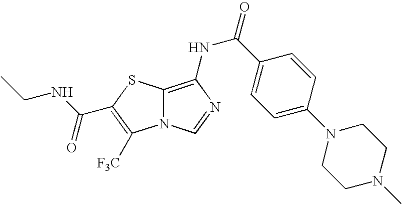 | 481.5 |
| 7-{[4-(4-methylpiperazin-1- yl)benzoyl]amino}-N-(2,2,2- trifluoroethyl)-3- (trifluoromethyl)imidazo[5,1- b][1,3]thiazole-2- carboxamide | 30 | 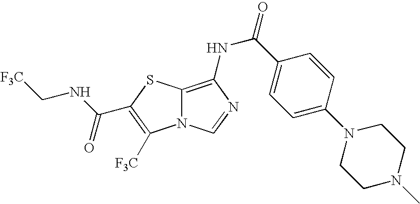 | 535.6 |
| N,N-dimethyl-7-{[4-(4- methylpiperazin-1- yl)benzoyl]amino}-3- (trifluoromethyl)imidazo [5,1- b][1,3]thiazole-2- carboxamide | 31 | 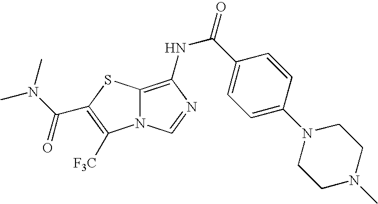 | 481.5 |

1. A compound of formula I: 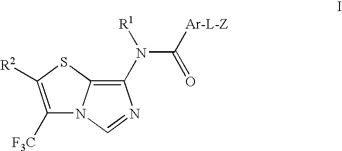
or a pharmaceutically acceptable salt or hydrate thereof; wherein:
R¹ represents H or C₁₋₄alkyl;
R² represents H, halogen C₁₋₄alkyl or CON(R⁴)₂;
each R⁴ independently represents H or C₁₋₄alkyl which is optionally substituted with up to 3 fluorine atoms;
Ar represents phenyl, naphthyl or heteroaryl of up to 10 ring atoms and is optionally substituted with halogen or C₁₋₄alkyl;
L represents a bond or a linking group represented by (X)ₘ—(CH₂)ₙ—(Y)ₚ;
m and p each independently is 0 or 1;
n is 0, 1, 2 or 3 but n is not 0 if m and p each represents 1;
X and Y independently represent O or NR³;
Z represents H, halogen, CF₃, CN, COR³, CO₂R³, CON(R³)₂, C₃₋₆cycloalkyl, phenyl, naphthyl or heterocyclyl of up to 10 ring atoms, said cycloalkyl, phenyl, naphthyl or heterocyclyl optionally bearing up to 2 substituents selected from halogen, C₁₋₄alkyl, CF₃, OH and C₁₋₄alkoxy;
with the proviso that when Z represents halogen or CN, L represents (X)ₘ—(CH₂)_{q} where q is 1, 2 or 3; and
R³ represents H or C₁₋₄alkyl; or two R³ groups attached to the same nitrogen atom may complete an N-heterocyclyl group of up to 10 ring atoms which optionally bears up to 2 substituents selected from halogen, C₁₋₄alkyl, CF₃ OH and C₁₋₄alkoxy.

2. A compound according to claim 1 wherein Ar represents phenyl or 6-membered heteroaryl.

3. A compound according to claim 1 wherein Ar represents 5-membered heteroaryl.

4. A compound according to claim 1 wherein L is selected from a bond, O, NH, CH₂, OCH₂CH₂ and NHCH₂CH₂.

5. A compound according to claim 1 wherein Z represents H, halogen, CN, COR³, CO₂R³ or optionally substituted phenyl or heterocyclyl.

6. A compound according to claim 1 wherein the moiety L-Z is selected from the group consisting of: H, CN, methoxy, Cl, F, CHO, CO₂Me, OH, phenoxy, piperazin-1-yl, 4-methylpiperazin-1-yl, 2-(piperidin-1-yl)ethoxy, morpholin-4-yl, hydroxymethyl, (4-methylpiperazin-1-yl)methyl, (piperidin-1-yl)methyl, (morpholin-4-yl)methyl and N-(1-methylpiperidin-4-yl)aminomethyl.

7. A compound according to claim 1 of formula II: 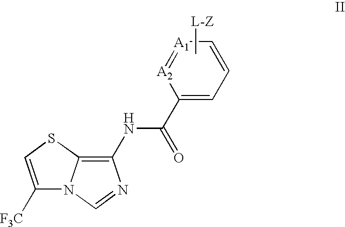
and pharmaceutically acceptable salts or hydrates thereof,
wherein A₁ and A₂ each represents CH or N but do not both represent N; and L and Z are as defined in claim 1.

8. A compound according to claim 7 wherein A₂ is CH.

9. A pharmaceutical composition comprising a compound according to claim 1 and a pharmaceutically acceptable carrier.

10. (canceled)

11. A method for treatment or prevention of a neurodegenerative disease associated with hyperphosphorylation of tau in a human patient, said method comprising administering to that patient an effective amount of a compound of formula I as defined in claim 1, or a pharmaceutically acceptable salt or hydrate thereof.
